(19) United States
(12) Patent Application Publication
(10) Pub. No.: US 2012/0302909 A1
(43) Pub. Date: Nov. 29, 2012

(54) METHODS AND SYSTEMS FOR SCREENING SUBJECTS

(76) Inventors: Martin L. Mayse, University City, MO (US); Steven C. Dimmer, Bellevue, WA (US)

(21) Appl. No.: 13/509,582

(22) PCT Filed: Nov. 11, 2010
(86) PCT No.: PCT/US10/56425

§ 371 Date: Aug. 4, 2012

Related U.S. Application Data
(60) Provisional application No. 61/260,352, filed on Nov. 11, 2009.

(51) Int. Cl. A61B 5/08; A61B 5/091; A61K 49/00

(52) U.S. Cl. 600/532; 424/9.1; 600/538; 600/529

(57) ABSTRACT

A screening method can be used to determine whether a subject is a suitable candidate for interventional therapy. The method can be used to determine the likelihood the subject will receive a therapeutic effect from denervation therapy. The determination is based, at least in part, on lung information obtained by performing lung function tests with and without treating the subject's lungs with a test agent. Based on the response to a test agent, the subject's response to a therapy is predicted.

### CROSS-REFERENCE TO RELATED APPLICATION

This application claims the benefit under 35 U.S.C. §119(e) of U.S. Provisional Patent Application No. 61/260,352 filed Nov. 11, 2009. This provisional application is incorporated herein by reference in its entirety.

### BACKGROUND

1. Technical Field

The present invention generally relates to evaluating subjects with respiratory diseases to determine whether a therapy will be therapeutically effective.

2. Description of the Related Art

Pulmonary diseases may cause a wide range of problems that adversely affect performance of the lungs. Pulmonary diseases, such as asthma and chronic obstructive pulmonary disease (“COPD”), may lead to increased airflow resistance in the lungs. Mortality, health-related costs, and the size of the population having adverse effects due to pulmonary diseases are all substantial. These diseases often adversely affect quality of life. Symptoms are varied but often include cough, breathlessness, and wheeze. In COPD, for example, breathlessness may be noticed when performing somewhat strenuous activities, such as running, jogging, brisk walking, etc. As the disease progresses, breathlessness may be noticed when performing non-strenuous activities, such as walking. Over time, symptoms of COPD may occur with less and less effort until they are present all of the time, thereby severely limiting a person's ability to accomplish normal tasks.

### BRIEF SUMMARY

In some embodiments, a screening method is used to identify appropriate candidates for a particular therapy. The method can be used to assure that treated subjects will potentially receive significant or full therapeutic effects. The screening method also assures that subjects who would not receive a therapeutic effect at or above a threshold level are not exposed to potential risks inherent with the therapy. For the majority of subjects with respiratory diseases, lung denervation will likely improve respiratory function and/or exercise capacity. However, some subjects may not receive therapeutic benefits that justify performing lung denervation therapy. Screening can be used to determine the potential effectiveness of lung denervation therapy, potential adverse affects (if any), or the like. Subjects with a high likelihood of receiving a therapeutic effect at or above a threshold level are identified as candidates for interventional therapy. Other subjects are identified as non-candidates. Although a non-candidate may be made aware of the inherent risks and relatively low likelihood of receiving a therapeutic effect, the non-candidate may elect to pursue the denervation procedure.

Lung denervation therapy can effectively treat different respiratory diseases (e.g., emphysema, chronic bronchitis, and asthma) and can involve damaging nerve tissue to substantially prevent nervous system signals from traveling to distal bronchial branches connected to the treatment site. In some procedures, the method can prevent nervous system signals from traveling to substantially all distal bronchial branches connected to the bronchus treatment site. Nerve trunks which traverse along the outside of both the right and left main bronchi can be ablated to effectively disconnect the vagus nerve and airway smooth muscle which lines the inside of the lung airways and also mucus producing glands located within the airways. When this occurs, airway smooth muscle relaxes and mucus production is decreased. These changes reduce airway obstruction. Reduced airway obstruction makes breathing easier, which can improve a subject's quality of life and health status.

In some embodiments, subjects are screened by using anticholinergic agents which temporarily block nerve signals to airway smooth muscle to relax the smooth muscle and open obstructed airways. Based at least in part on the subject's response to the agents, a physician can determine a predicted response to lung denervation therapy. For example, subjects that respond to anticholinergic agents may likely be responsive to lung denervation therapy. Non-responsiveness to the agents may indicate that a subject will not be responsive to lung denervation therapy.

A method for evaluating a subject, in some embodiments, is a non-therapeutic method that comprises acquiring a first pulmonary test result or set of data for the subject's lungs from a first lung function test. A test agent is administered to the subject. A second pulmonary test result or set of data is acquired for the subject's treated lungs. The second pulmonary test result or set of data is acquired using a second lung function test. The first and second test results or sets of data can be analyzed to evaluate whether the subject is suitable for therapy.

The test results or data can include one or more of forced expiratory volume in one second, forced vital capacity, total lung capacity, airflow resistance, or other measurements capable of being acquired using a spirometer or other respiratory testing equipment. The test results or data can be compared to categorize the subject. To categorize for denervation therapy, one group can be a candidate group comprised of subjects suitable for denervation therapy. A non-candidate group can be comprised of subjects that are not suitable for denervation therapy. Non-candidates can be individuals that are non-responsive to anticholinergic agents. A physician or computing system can categorize the subjects.

In other embodiments, a screening method includes evaluating a subject's baseline lung function. The subject's lung function is also evaluated using a bronchial challenge test. The subject's respiratory system treated with a therapeutic agent is evaluated. Test results are compared to determine whether to perform interventional therapy. In some embodiments, the subject can be identified as a candidate for interventional therapy or a non-candidate for interventional therapy.

In yet further embodiments, a method is a non-therapeutic method that of evaluating a subject comprises acquiring first pulmonary test results for the subject's lungs from a first lung function test performed on the subject. Second pulmonary test results for the subject's lungs are acquired when the respiratory system is treated with an agent. The second pulmonary test results are obtained using a second lung function test performed on the subject. It is determined whether the subject's respiratory response to a test agent meets at least one acceptance criterion based, at least in part, on an evaluation of the first and second pulmonary test results. In particular embodiments, the acceptance criterion corresponds to denervation therapy and is in the form of a responsiveness threshold level. If the subject's lung function improves to a threshold level (e.g., a predetermined threshold level), the respiratory response meets the acceptance criterion. In certain protocols, if FEV1 increases by 10% in response to an agent (e.g., an anticholinergic agent), the subject's response meets an acceptance criterion comprising a threshold level of lung function improvement of a 10% increase of FEV1 threshold levels for other lung test values can also be used.

In some other embodiments, a system for evaluating a subject comprises a storage device and a computing system. The storage device can store test results. The computing system is configured to execute a program to compare the stored test results and to categorize subjects based on the comparison. The computing system, in some embodiments, categorizes based on whether lung function improves to a threshold level in response to an administered agent. The computing system can also retrieve test results to generate a treatment program, to compare test results, and to create patient records (e.g., physical records, electronic records, or the like), or the like. The treatment procedure can be a denervation procedure.

At least some denervation procedures include moving a catheter along a lumen of an airway of a bronchial tree. The airway includes a first tubular section, a second tubular section, a treatment site between the first tubular section and the second tubular section, and a nerve extending along at least the first tubular section, the treatment site, and the second tubular section. The nerve can be within or outside of the airway wall. In some embodiments, the nerve is a nerve trunk outside of the airway wall and connected to a vagus nerve.

The catheter can damage a portion of the nerve at the treatment site to substantially prevent signals from traveling between the first tubular section and the second tubular section via the nerve. In some embodiments, blood flow between the first tubular section and the second tubular section can be maintained while damaging a portion of the nerve. The continuous blood flow can maintain desired functioning of distal lung tissue.

The second tubular section of the airway may dilate in response to the damage to the nerve. Because nervous system signals are not delivered to smooth muscle of the airway of the second tubular section, smooth muscle can relax so as to cause dilation of the airway, thereby reducing airflow resistance, even airflow resistance associated with pulmonary diseases. In some embodiments, nerve tissue can be damaged to cause dilation of substantially all the airways distal to the damaged tissue. The nerve can be a nerve trunk, nerve branch, nerve fibers, and/or other accessible nerves.

In some embodiments, a method for treating a subject includes moving an intraluminal device, such as a catheter, along a lumen of an airway of a bronchial tree. A portion of the airway is denervated using the intraluminal device. In some embodiments, the portion of the airway is denervated without irreversibly damaging to any significant extent an inner surface of the airway. In some embodiments, a portion of a bronchial tree is denervated without irreversibly damaging to any significant extent nerve tissue (e.g., nerve tissue of nerve fibers) within the airway walls of the bronchial tree. The inner surface can define the lumen along which the intraluminal device was moved.

The denervating process can be performed without destroying at least one artery extending along the airway. In some embodiments, substantially all of the arteries extending along the airway are preserved during the denervating process. In some embodiments, one or more nerves embedded in the wall of the airway can be generally undamaged during the denervating process. The destroyed nerves can be nerve trunks outside of the airway.

In some embodiments, the denervating process can decrease smooth muscle tone of the airway to achieve a desired increased airflow into and out of the lung. In some embodiments, the denervating process causes a sufficient decrease of smooth muscle tone so as to substantially increase airflow into and out of the lung. For example, the subject may have an increase in FEV1 of at least 10% over a baseline FEV1. As such, the subject may experience significant improved lung function when performing normal everyday activities, even strenuous activities. In some embodiments, the decrease of airway smooth muscle tone is sufficient to cause an increase of FEV1 in the range of about 10% to about 30%. Any number of treatment sites can be treated either in the main bronchi, segmental bronchi or subsegmental bronchi to achieve the desired increase in lung function.

In some embodiments, an elongate assembly for treating a lung is adapted to damage nerve tissue of a nerve trunk so as to attenuate nervous system signals transmitted to a more distal portion of the bronchial tree. The tissue can be damaged while the elongated assembly extends along a lumen of the bronchial tree. A delivery assembly can be used to provide access to the nerve tissue.

In some other embodiments, a system for treating a subject includes an elongate assembly dimensioned to move along a lumen of an airway of a bronchial tree. The elongate assembly is adapted to attenuate signals transmitted by nerve tissue, such as nerve tissue of nerve trunks, while not irreversibly damaging to any significant extent an inner surface of the airway. The elongate assembly can include an embeddable distal tip having at least one actuatable element, such as an ablation element. The ablation element can ablate various types of nerve tissue when activated. In some embodiments, the ablation element includes one or more electrodes operable to output radiofrequency energy.

In some embodiments, a method comprises damaging nerve tissue of a first main bronchus to substantially prevent nervous system signals from traveling to substantially all distal bronchial branches connected to the first main bronchus. In some embodiments, most or all of the bronchial branches distal to the first main bronchus are treated. The nerve tissue, in certain embodiments, is positioned between a trachea and a lung through which the bronchial branches extend. The method further includes damaging nerve tissue of a second main bronchus to substantially prevent nervous system signals from traveling to substantially all distal bronchial branches connected to the second main bronchus. A catheter assembly can be used to damage the nerve tissue of the first main bronchus and to damage the nerve tissue of the second main bronchus without removing the catheter assembly from a trachea connected to the first and second bronchi.

In some embodiments, a method comprises denervating most of a portion of a bronchial tree to substantially prevent nervous system signals from traveling to substantially all bronchial branches of the portion. In certain embodiments, denervating procedures involve damaging nerve tissue using less than about 100 applications of energy, 50 applications of energy, 36 applications of energy, 18 applications of energy, 10 applications of energy, or 3 applications of energy. Each application of energy can be at a different treatment site. In some embodiments, substantially all bronchial branches in one or both lungs are denervated by the application of energy.

In yet further embodiments, a bronchial challenge test can be performed on a subject. The bronchial challenge test can involve using one or more therapeutic agents (e.g., agents that cause bronchial constriction), electrical stimulation, nerve stimulation, or other types of techniques suitable for closing or constricting airways. In some bronchial challenge tests, one or more agents are delivered to the subject's respiratory system. In electrical stimulation embodiments, electrical stimulation can be used to close airways. Therapeutic agents can also be utilized in combination with electrical stimulation. In yet further protocols, agents can be used for one part of the bronchial challenge test and electrical stimulation can be used to perform another part of the bronchial challenge test. Thus, agents, stimulation (e.g., electrical stimulation), and other techniques can be used, alone or in combination, to perform one or more bronchial challenge tests or other tests.

### BRIEF DESCRIPTION OF THE SEVERAL VIEWS OF THE DRAWINGS

FIG. 1 is a flow chart of a process for screening subjects.

FIG. 2 is an illustration of lungs, blood vessels, and nerves near to and in the lungs.

FIG. 3 is a schematic view of a treatment system positioned within a left main bronchus according to one embodiment.

FIG. 4 is a flow chart of a process for screening subjects.

FIG. 5 is a flow chart of another process for screening subjects.

FIG. 6 illustrates a system for screening subjects.

### DETAILED DESCRIPTION

Therapies for treating a respiratory system may effectively treat many diseases for certain individuals but may not be suitable for other individuals. It may be difficult to determine which individuals will respond to the therapies. There is often a wide range of efficacy, including efficacy levels for some subjects that may not justify the procedural risk associated with the therapy. A screening method can be used to determine the likelihood a subject will experience a desired therapeutic response prior to exposing the subject to risks inherent with the therapy and can be used to categorize the subjects into different groups (e.g., a group recommended for therapy, a group not recommend for therapy, a group for monitoring, etc.). Screening can be performed for different types of therapies, including lung denervation therapy, bronchial thermoplasty, lung resection, intrabronchial valve therapy, smooth muscle relaxation therapy, or the like, to predict therapy efficacy, to assess potential adverse effects, to evaluate ancillary benefits of therapy, or the like.

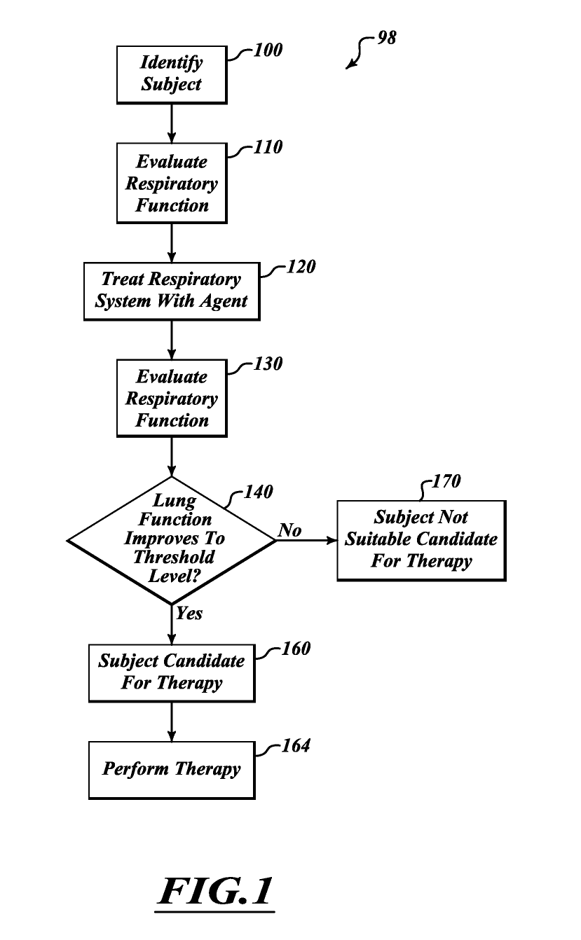

FIG. 1 is a flow chart of a screening method 98 for determining whether a subject is likely to respond favorably to a therapy. Generally, a subject with a respiratory disease can be identified at 100. At 110, respiratory function is evaluated. Baseline lung function can be analyzed. At 120, an agent for altering lung function is administered. At 130, the subject is evaluated to assess the subject's response to the agent. At 140, if the subject's lung function improves in response to the agent to a threshold level, the subject is identified as a candidate for therapy. If lung function improvement, if any, does not reach the threshold level, the subject is identified as not being a suitable candidate at 170. Additional tests can also be performed to acquire information about the subject. At 164, a therapy can be performed. The screening method and therapies are detailed below.

At 100, subjects are identified for the screening process and may be diagnosed with a respiratory disease. Pulmonary function tests can be used to diagnose respiratory diseases. By way of example, forced expiratory volume in 1 second (FEV1) and forced vital capacity (FVC) can be used to diagnose COPD. A ratio of FEV1/FVC less than 0.7 after the administration of at least one bronchodilator defines the presence of COPD. In some protocols, symptomatic subjects can be tested to obtain lung function information that is compared to predicted lung function. A subject with an FEV1 less than a percentage (e.g., 50%, 70%, 80%, or 90%) of predicted FEV1 can be identified for the screening process.

Pulmonary diseases can also be diagnosed using imaging or direct visualization (e.g., bronchoscopy) because the diseases are often characterized by visible airway obstructions associated with blockage of an airway lumen, thickening of an airway wall, alteration of structures within or around the airway wall, or combinations thereof. Airway obstruction can significantly decrease the amount of gas exchanged in the lungs and often results in breathlessness. Blockage of an airway lumen can be caused by excessive intraluminal mucus or edema fluid, or both. Thickening of the airway wall may be attributable to excessive contraction of the airway smooth muscle, airway smooth muscle hypertrophy, mucous glands hypertrophy, inflammation, edema, or combinations thereof. Alteration of structures around the airway, such as destruction of the lung tissue itself, can lead to a loss of radial traction on the airway wall and subsequent narrowing of the airway.

Asthma can be characterized by contraction of airway smooth muscle, smooth muscle hypertrophy, excessive mucus production, mucous gland hypertrophy, and/or inflammation and swelling of airways. These abnormalities are the result of a complex interplay of local inflammatory cytokines (chemicals released locally by immune cells located in or near the airway wall), inhaled irritants (e.g., cold air, smoke, allergens, or other chemicals), systemic hormones (chemicals in the blood such as the anti-inflammatory cortisol and the stimulant epinephrine), local nervous system input (nerve cells contained completely within the airway wall that can produce local reflex stimulation of smooth muscle cells and mucous glands), and the central nervous system input (nervous system signals from the brain to smooth muscle cells and mucous glands carried through the vagus nerve). These conditions often cause widespread temporary tissue alterations and initially reversible airflow obstruction that may ultimately lead to permanent tissue alteration and permanent airflow obstruction that make it difficult for the asthma sufferer to breathe. Asthma can further include acute episodes or attacks of additional airway narrowing via contraction of hyper-responsive airway smooth muscle that significantly increases airflow resistance. Asthma symptoms include recurrent episodes of breathlessness (e.g., shortness of breath or dyspnea), wheezing, chest tightness, and cough.

Emphysema is a type of COPD often characterized by the alteration of lung tissue surrounding or adjacent to the airways in the lungs. Emphysema can involve destruction of lung tissue (e.g., alveoli tissue such as the alveolar sacs) that leads to reduced gas exchange and reduced radial traction applied to the airway wall by the surrounding lung tissue. The destruction of alveoli tissue leaves areas of emphysematous lung with overly large airspaces that are devoid of alveolar walls and alveolar capillaries and are thereby ineffective at gas exchange. Air becomes “trapped” in these larger airspaces. This “trapped” air may cause over-inflation of the lung, and in the confines of the chest restricts the in-flow of oxygen rich air and the proper function of healthier tissue. This results in significant breathlessness and may lead to low oxygen levels and high carbon dioxide levels in the blood. This type of lung tissue destruction occurs as part of the normal aging process, even in healthy individuals. Unfortunately, exposure to chemicals or other substances (e.g., tobacco smoke) may significantly accelerate the rate of tissue damage or destruction. Breathlessness may be further increased by airway obstruction. The reduction of radial traction may cause the airway walls to become “floppy” such that the airway walls partially or fully collapse during exhalation. An individual with emphysema may be unable deliver air out of their lungs due to this airway collapse and airway obstructions during exhalation. Lung denervation and intrabonchial valve therapy may be especially well suited to treat emphysema.

Chronic bronchitis is a type of COPD that can be characterized by contraction of the airway smooth muscle, smooth muscle hypertrophy, excessive mucus production, mucous gland hypertrophy, and inflammation of airway walls. Like asthma, these abnormalities are the result of a complex interplay of local inflammatory cytokines, inhaled irritants, systemic hormones, local nervous system, and the central nervous system. Unlike asthma where respiratory obstruction may be largely reversible, the airway obstruction in chronic bronchitis is primarily chronic and permanent. It is often difficult for a chronic bronchitis sufferer to breathe because of chronic symptoms of shortness of breath, wheezing, and chest tightness, as well as a mucus producing cough. Bronchodilators can be used to open the airways to temporarily reduce respiratory resistance. Lung denervation may be especially well suited to treat chronic bronchitis.

At 110, the subject's respiratory function is evaluated. Tests can be administered at a hospital, a clinic, or other appropriate setting to obtain baseline lung function information. The lung function information can include, without limitation, one or more lung function test results (including measurements or data), questionnaire scores, and observations (e.g., a physician's observations). Pulmonary function tests, blood gases tests, exercise capacity tests, and questionnaires can be used to obtain such information and are discussed below.

Pulmonary function tests can provide objective and reproducible measures of basic physiologic lung parameters, such as total airflow, lung volume, and gas exchange. Indices of pulmonary function tests used for the assessment of pulmonary diseases, especially obstructive pulmonary diseases, include the forced expiratory volume in 1 second (FEV1), the forced vital capacity (FVC), the ratio of the FEV1 to FVC, the total lung capacity (TLC), airway resistance, and the testing of arterial blood gases. FEV1 is the volume of air a patient can exhale during the first second of a forceful exhalation which starts with the lungs completely filled with air. FEV1 is also the average flow that occurs during the first second of a forceful exhalation. The FVC is the total volume of air a patient can exhale during a forceful exhalation that starts with the lungs completely filled with air. The FEV1/FVC is the fraction of all the air that can be exhaled during a forceful exhalation during the first second. TLC is the total amount of air within the lungs when the lungs are completely filled and may increase when air becomes trapped within the lungs of subjects with obstructive lung disease. Airway resistance is defined as the pressure gradient between the alveoli and the mouth to the rate of airflow between the alveoli and the mouth. similarly, resistance of a given airway would be defined as the ratio of the pressure gradient across the given airway to the flow through the airway. The pulmonary function tests can be used to obtain results for baseline lung function and lung function associated with administered therapeutic agent(s).

Arterial blood gases tests measure the amount of oxygen and the amount of carbon dioxide in the blood and are the most direct method for assessing the ability of the lungs and respiratory system to bring oxygen from the air into the blood and to get carbon dioxide from the blood out of the body. Arterial blood gases tests can provide accurate and objective measurement of lung function.

Exercise capacity tests can provide reproducible measures of a subject's ability to perform activities. A six minute walk test (6MWT) is an exercise capacity test in which a subject walks as far as possible over a flat surface in 6 minutes. Another exercise capacity test involves measuring the maximum exercise capacity of a subject. For example, a physician or physician assistant can measure the amount of power the subject can produce while on a cycle ergometer. The subject can breathe 30 percent oxygen and the work load can increase by about 5 to about 10 watts every 3 minutes.

Questionnaires assess a subject's overall health and well being. The St. George's Respiratory Questionnaire is a quality of life questionnaire that includes 75 questions designed to measure the impact of obstructive lung disease on overall health, daily life, and perceived well-being. The efficacy of a treatment for pulmonary diseases can be evaluated using pulmonary function tests, exercise capacity tests, and/or questionnaires.

Baseline results when the subject's lungs are not treated with any therapeutic agents can be obtained. The results can include, without limitation, a raw data, a set of measurements, calculated results (e.g., ratios such as a FEV1/FVC ratio), graphs, or combinations thereof. Composite respiratory function values can be generated using results from pulmonary function tests, results from exercise capacity tests, information from questionnaires, or the like. Test results or scores may be weighted differently based on, for example, the subject's health status. For example, if the subject appears to exert minimal effort during an exercise capacity test, the pulmonary function test results or blood gases test results may be weighted more than the results from the exercise capacity test.

Respiratory testing equipment for pulmonary function testing can include one or more spirometers, ventilators, respirators, breathing machines, airflow sensors, respiratory masks, or the like. Spirometers can include, without limitation, volume displacement spirometers, flow-sensing spirometers, windmill-type spirometers. A spirometer can be used to obtain FEV1, forced expiratory volume in the first six seconds (“FEV6”), or FVC, as well as other lung function tests or measures associated with pulmonary function, such as respiratory pressures (e.g., maximum expiratory pressures).

A health care provider can have a computing system in communication with the respiratory testing equipment. The computing system can analyze and store information and can include, without limitation, one or more computers, servers, internet based computer systems, local computer systems, central processing units, microprocessors, and storage devices (e.g., hard drives, storage mediums, storage disks, memory, storage elements, or the like). In some embodiments, the computing system has circuitry configured to generate a treatment protocol based on one or more comparisons of stored first test results and stored second test results. For example, the circuitry can be configured to generate a lung denervation protocol. The denervation protocol can include, without limitation, targeted treatment sites, energy doses (e.g., RF energy doses for RF ablation), instrument settings, and other treatment parameters. Different types of optimization programs can be executed by the computing system. Programs (e.g., programs stored in memory) can be used to compare, correlate, generate graphs, predict lung function, generate treatment programs, or the like. Different programs can correspond to different diseases. For example, one program can be executed for subjects with asthma, and another program can be executed for subjects with COPD.

At 120, at least one agent is administered to the subject. The dose can be selected based on the subject's age, weight, diagnosis, symptoms, and other criteria. Agents can be administered systemically (e.g., intravenously), orally, by inhalation, or the like. A physician can administer the agent systematically. An inhaler can be used by the subject for self-administration. An inhaler can include an array of pre-metered dosages of the agent to ensure that a desired predicted therapeutically effective amount of the agent is administered to the subject. For example, ipratropium bromide can be inhaled by the subject. The dose can be in a range of about 17 mcg to about 34 mcg. For severe cases of COPD, the dose can be in a range of about 17 mcg to about 136 mcg. Other doses can be used, if needed or desired.

The agent can be a test agent comprising an anticholinergic agent, nerve signal blocking agent, or other type of agent. The anticholinergic agent can be an inhalable neurotransmitter or substance that is antagonistic to the action of parasympathetic or other cholinergic nerve fibers. Anticholinergic agents can include, without limitation, a bronchodilator, atropine, ipratropium bromide (Atrovent®, Apovent®, Aerovent®), oxitropium bromide (Oxivent®), and tiotropium (Spiriva®). Nerve signal blocking agents that temporarily block nerve signals include, without limitation, lidocaine (Xylocalne®), mepivacaine (Carbocaine®), bupivacaine (Marcaine®), prilocalne (Citanest®), procaine (Novacaine®), ropivacaine (Naropine®), and tetracaine (Pontocaine®). In some embodiments, a beta2-agonist (e.g., short-acting or long-acting) in combination with an anticholinergic bronchodilator is administered.

At 130, respiratory function is evaluated to determine the subject's response, if any, to the agent. For convenient comparison, the same tests can be performed at 110 and 130. If pulmonary tests measure FEV1 and FVC at 110, FEV1 and FVC can be measured at 130. Additional tests can be used to monitor respiratory function with and without using the agent. For example, respiratory function can be monitored over a length of time (e.g., about 15 minutes to about 2 hours after administering an agent) which the administered agent is likely to be effective.

At 140, test results from the tests at 110, 130 are analyzed to determine whether lung function meets one or more acceptance criteria. One criterion is whether lung function has improved to a therapeutic threshold level. If the subject's response to the agent meets the acceptance criterion, the subject is identified as a candidate for therapy. If lung function has improved to or by a therapeutic threshold level, then the subject can be identified as a suitable candidate for interventional therapy at 160. The therapeutic threshold level can be a therapeutic increase in FEV1, FVC, lung capacity, or other values, and can be based on an absolute value (e.g., whether a measured value is at or above a predetermined value), differences between values, relative changes in values, or the like. In some embodiments, a set of criteria is used to evaluate the test results to determine if the subject's lung function improves to a threshold level.

If FEV1 measured at 130 is at least a threshold percentage (e.g., 10%, 15%, 20%, or 30%) greater than the FEV1 measured at 110, the subject can be identified as a candidate for interventional therapy. In some protocols, if FEV1 at 130 is at least 20% greater than FEV1 at 110, the subject meets a 20% increase in FEV1 criterion and is selected for interventional therapy. Alternatively, FEV1 measured at 130 is at least a threshold absolute improvement (e.g., 200 ml) greater than the FEV1 measured at 110, the subject can be identified as a candidate for interventional therapy. Alternatively, a combination of threshold percentage and absolute improvement (e.g., 12% and 200 ml) may be used. By way of another example, the threshold level for an exercise capacity test can be 110% of a baseline six minute walk test. If the subject walks about 0.5 miles at 110 and more than 0.55 miles at 130, the subject has shown lung function that exceeds a therapeutic threshold level. Alternatively, an absolute improvement measured at 130 (e.g., 177 feet or 54 meters), regardless of the percentage change compared to 110, may indicate that subject exceeds a therapeutic threshold level. Data acquired using pulmonary function tests can be weighted and combined for a total pulmonary test score. The total pulmonary test score from 110 can be compared to a corresponding total pulmonary test score from 130. Based on a difference between the scores, a ratio of the scores, or other relationship between the scores, it can be determined whether the agent caused a threshold improvement in lung function. A physician can determine a threshold level as desired.

At 160, the subject can be identified as a candidate and, in some embodiments, can be categorized into a candidate group or a non-candidate group. If the subject's medical information is stored on a computing system, the subject's information can be changed to indicate group status. The subject can also be informed whether he or she will likely respond to interventional therapy.

At 164, a procedure can be performed on the subject. The procedure can include, without limitation, denervation (e.g., denervation of the bronchial tree), bronchial thermoplasty, implanting an intrabronchial valve or stent, or performing other types of procedures that may alter or isolate at least a portion of the lungs. If the subject responded to the test agent, a similar response may be achieved by total lung denervation. Regional lung denervation can also be performed if isolated regions of a lung or bronchial tree are diseased. The denervation procedure may have a therapeutic effectiveness that is generally proportional to the therapeutic effectiveness of the test agent. In some procedures, nerve tissue of a nerve trunk extending along the airway of the bronchial tree can be damaged to attenuate nervous system signals transmitted to a portion of the bronchial tree.

The results from pulmonary test functions can be used to select pulmonary therapy. For example, a subject with COPD and a FEV1 equal to or less than 50% of predicted FEV1 may be screened for a denervation at both lungs. A subject with COPD and FEV1 in a range of 50% to 80% of predicted FEV1 may be identified as a candidate for lung denervation of one lung only. Subjects may also be identified using questionnaires (e.g., symptom based questionnaires) and/or a lung capacity test. Other tests can be utilized depending on whether the physician believes that the subject has pulmonary disease, asthma, emphysema, chronic bronchitis, or the like.

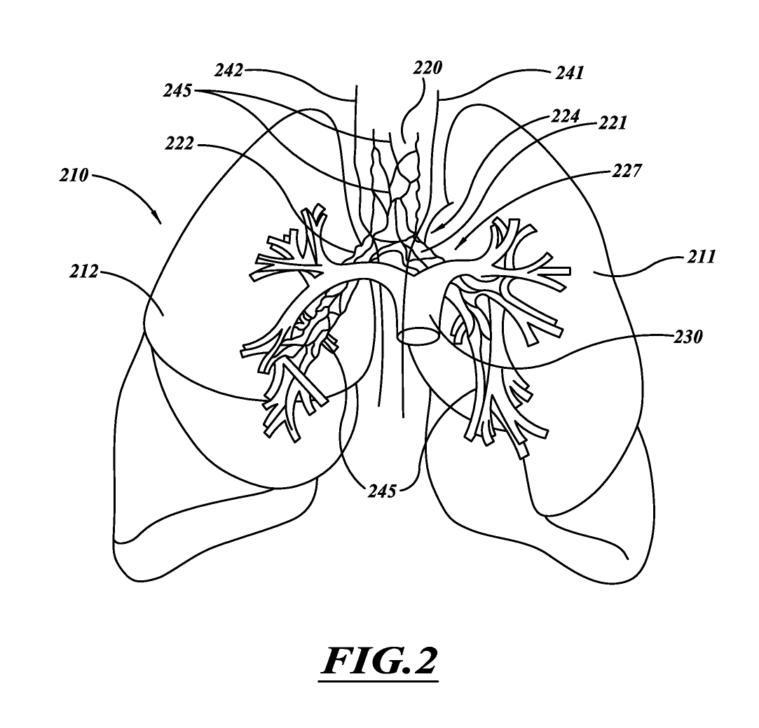

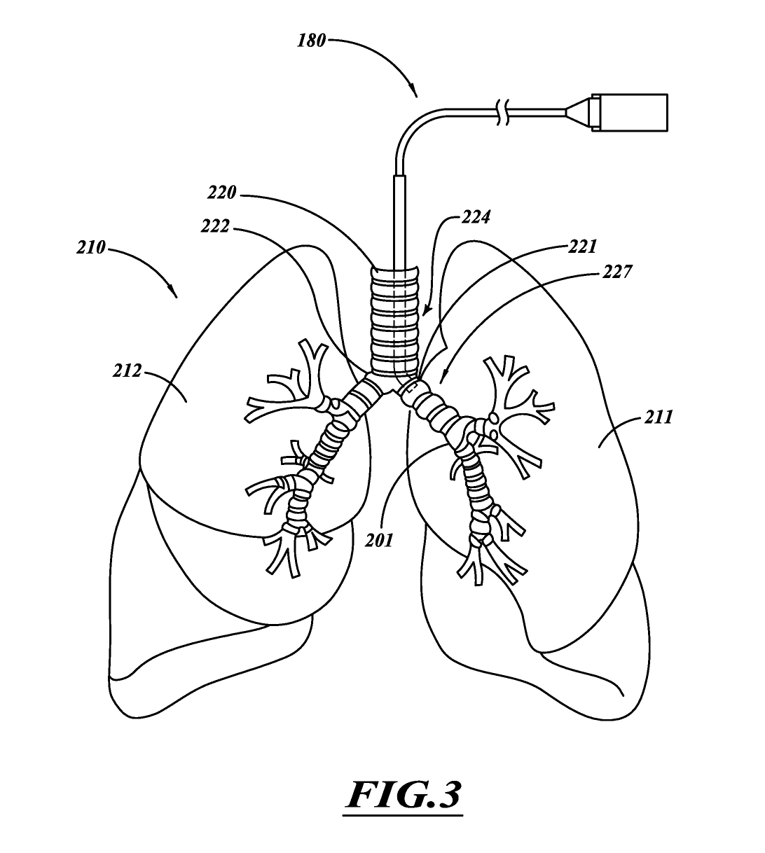

Denervation procedures are disclosed in U.S. application Ser. No. 12/463,304 filed May 8, 2009 and U.S. application Ser. No. 12/913,702 filed Oct. 27, 2010. The two co-pending applications are incorporated by reference in their entireties and also disclose systems, apparatuses (e.g., catheters, elongate assemblies, etc.), and ablation denervation procedures. In some denervation procedures, at least one nerve trunk adjacent to an airway wall is destroyed to denervate a substantial portion of the left or right lung. Such procedures may cause ancillary damage or may pose certain risks. The screening method can be used to assess whether the potential therapeutic effectiveness of the denervation process justifies the associated risks. Denervation procedures are discussed in connection with FIGS. 2 and 3.

FIG. 2 illustrates human lungs 210 having a left lung 211 and a right lung 212. A trachea 220 extends downwardly from the nose and mouth and divides into a left main bronchus 221 and a right main bronchus 222. The left main bronchus 221 and right main bronchus 222 each branch to form lobar, segmental bronchi, and sub-segmental bronchi, which have successively smaller diameters and shorter lengths in the outward direction (i.e., the distal direction). A main pulmonary artery 230 originates at a right ventricle of the heart and passes in front of a lung root 224. At the lung root 224, the artery 230 branches into a left and right pulmonary artery, which in turn branch to form a network of branching blood vessels. These blood vessels can extend alongside airways of a bronchial tree 227. The bronchial tree 227 includes the left main bronchus 221, the right main bronchus 222, bronchioles, and alveoli. Vagus nerves 241, 242 extend alongside the trachea 220 and branch to form nerve trunks 245.

The left and right vagus nerves 241, 242 originate in the brainstem, pass through the neck, and descend through the chest on either side of the trachea 220. The vagus nerves 241, 242 spread out into nerve trunks 245 that include the anterior and posterior pulmonary plexuses that wrap around the trachea 220, the left main bronchus 221, and the right main bronchus 222. The nerve trunks 245 also extend along and outside of the branching airways of the bronchial tree 227. Nerve trunks 245 are the main stem of a nerve, comprising a bundle of nerve fibers bound together by a tough sheath of connective tissue.

The prime function of the lungs 210 is to exchange oxygen from air into the blood and to exchange carbon dioxide from the blood to the air. The process of gas exchange begins when oxygen rich air is pulled into the lungs 210. Contraction of the diaphragm and intercostal chest wall muscles cooperate to decrease the pressure within the chest to cause the oxygen rich air to flow through the airways of the lungs 210. For example, air passes through the mouth and nose, the trachea 220, then through the bronchial tree 227. The air is ultimately delivered to the alveolar air sacs for the gas exchange process.

Oxygen poor blood is pumped from the right side of the heart through the pulmonary artery 230 and is ultimately delivered to alveolar capillaries. This oxygen poor blood is rich in carbon dioxide waste. Thin semi-permeable membranes separate the oxygen poor blood in capillaries from the oxygen rich air in the alveoli. These capillaries wrap around and extend between the alveoli. Oxygen from the air diffuses through the membranes into the blood, and carbon dioxide from the blood diffuses through the membranes to the air in the alveoli. The newly oxygen enriched blood then flows from the alveolar capillaries through the branching blood vessels of the pulmonary venous system to the heart. The heart pumps the oxygen rich blood throughout the body. The oxygen spent air in the lung is exhaled when the diaphragm and intercostal muscles relax and the lungs and chest wall elastically return to the normal relaxed states. In this manner, air can flow through the branching bronchioles, the bronchi 221, 222, and the trachea 220 and is ultimately expelled through the mouth and nose.

A treatment system 180 of FIG. 3 can be used to denervate the lungs 210 to adjust airflow during expiration or inhalation, or both. The treatment system 180 can include a catheter or elongate assembly with a distal tip 181 with one or more ablation elements 182 capable of outputting energy to destroy nerve tissue. Ablation elements can include, without limitation, radiofrequency electrodes, ultrasound emitters, microwave energy emitters, heating elements, ports (e.g., ports through which chemicals can be dispensed), cryogenic elements, or other elements capable of selectively damaging tissue. The distal tip 181 can be cooled using coolants to provide differential cooling, as disclosed in U.S. patent application Ser. Nos. 12/463,304 and 12/913,702. In certain embodiments, the distal tip 181 includes, without limitation, one or more expandable balloons, wire baskets, or other components capable of cooling airway walls to selectively ablate nerve tissue without damaging interior regions of the airway. For example, airways can be enlarged (e.g., dilated) to decrease airflow resistance and/or to increase gas exchange. Nerve tissue, such as nerve tissue of a nerve trunk, can be ablated by the ablation element 182 to dilate airways.

Different types of energy can be used to destroy targeted tissue. As used herein, the term “energy” is broadly construed to include, without limitation, thermal energy, cryogenic energy (e.g., cooling energy), electrical energy, acoustic energy (e.g., ultrasonic energy), radio frequency energy, pulsed high voltage energy, mechanical energy, ionizing radiation, optical energy (e.g., light energy), and combinations thereof, as well as other types of energy suitable for treating tissue. In some embodiments, the treatment system 180 delivers energy and also one or more substances (e.g., radioactive seeds, radioactive materials, etc.), treatment agents, and the like. Exemplary non-limiting treatment agents include, without limitation, one or more antibiotics, anti-inflammatory agents, pharmaceutically active substances, bronchoconstrictors, bronchodilators (e.g., beta-adrenergic agonists, anticholinergics, etc.), nerve blocking drugs, photoreactive agents, or combinations thereof. For example, long acting or short acting nerve blocking drugs (e.g., anticholinergics) can be delivered to the nerve tissue to temporarily or permanently attenuate signal transmission. Substances can also be delivered directly to the nerves or the nerve trunks, or both, to chemically damage the nerve tissue.

The treatment system 180 can target the nervous system which provides communication between the brain and the lungs 210 using electrical and chemical signals. A network of nerve tissue of the autonomic nervous system senses and regulates activity of the respiratory system and the vasculature system. Nerve tissue includes fibers that use chemical and electrical signals to transmit sensory and motor information from one body part to another. For example, the nerve tissue can transmit motor information in the form of nervous system input, such as a signal that causes contraction of muscles or other responses. The fibers can be made up of neurons. The nerve tissue can be surrounded by connective tissue, i.e., epineurium. The autonomic nervous system includes a sympathetic system and a parasympathetic system. The sympathetic nervous system is largely involved in “excitatory” functions during periods of stress. The parasympathetic nervous system is largely involved in “vegetative” functions during periods of energy conservation. The sympathetic and parasympathetic nervous systems are simultaneously active and generally have reciprocal effects on organ systems. While innervation of the blood vessels originates from both systems, innervation of the airways are largely parasympathetic in nature and travel between the lung and the brain in the right vagus nerve 242 and the left vagus nerve 241.

The treatment system 180 can perform any number of procedures on one or more of these nerve trunks 245 to affect the portion of the lung associated with those nerve trunks. Because some of the nerve tissue in the network of nerve trunks 45 coalesce into other nerves (e.g., nerves connected to the esophagus, nerves though the chest and into the abdomen, and the like), the treatment system 198 can treat specific sites to minimize, limit, or substantially eliminate unwanted damage of those other nerves. Some fibers of anterior and posterior pulmonary plexuses coalesce into small nerve trunks which extend along the outer surfaces of the trachea 220 and the branching bronchi and bronchioles as they travel outward into the lungs 210. Along the branching bronchi, these small nerve trunks continually ramify with each other and send fibers into the walls of the airways.

The treatment system 180 can affect specific nerve tissue, such as vagus nerve tissue, associated with particular sites of interest. Vagus nerve tissue includes efferent fibers and afferent fibers oriented parallel to one another within a nerve branch. The efferent nerve tissue transmits signals from the brain to airway effector cells, mostly airway smooth muscle cells and mucus producing cells. The afferent nerve tissue transmits signals from airway sensory receptors, which respond variously to irritants and stretch, to the brain. While efferent nerve tissue innervates smooth muscle cells all the way from the trachea 220 to the terminal bronchioles, the afferent fiber innervation is largely limited to the trachea 220 and larger bronchi. There is a constant, baseline tonic activity of the efferent vagus nerve tissues to the airways which causes a baseline level of smooth muscle contraction and mucous secretion.

The treatment system 180 can also affect the efferent and/or the afferent tissues to control airway smooth muscle (e.g., innervate smooth muscle) and mucous secretion. The contraction of airway smooth muscle and excess mucous secretion associated with pulmonary diseases often results in relatively high airflow resistance causing reduced gas exchange and decreased lung performance. Nerve tissue can be ablated to attenuate the transmission of signals traveling along the vagus nerves 241, 242 that cause muscle contractions, mucus production, and the like. Attenuation can include, without limitation, hindering, limiting, blocking, and/or interrupting the transmission of signals. For example, the attenuation can include decreasing signal amplitude of nerve signals or weakening the transmission of nerve signals. Decreasing or stopping nervous system input to distal airways can alter airway smooth muscle tone, airway mucus production, airway inflammation, and the like, thereby controlling airflow into and out of the lungs 210. In some embodiments, the nervous system input can be decreased to correspondingly decrease airway smooth muscle tone. In some embodiments, the airway mucus production can be decreased a sufficient amount to cause a substantial decrease in coughing and/or in airflow resistance. Signal attenuation may allow the smooth muscles to relax and prevent, limit, or substantially eliminate mucus production by mucous producing cells. In this manner, healthy and/or diseased airways can be altered to adjust lung function. After treatment, various types of questionnaires or tests can be used to assess the subject's response to the treatment. If needed or desired, additional procedures can be performed to reduce the frequency of coughing, decrease breathlessness, decrease wheezing, and the like.

Main bronchi 221, 222 (i.e., airway generation 1) of FIG. 2 can be treated to affect distal portions of the bronchial tree 227. In some embodiments, the left and right main bronchi 221, 222 are treated at locations along the left and right lung roots 224 and outside of the left and right lungs 211, 212. Treatment sites can be distal to where vagus nerve branches connect to the trachea and the main bronchi 221, 222 and proximal to the lungs 211, 212. A single treatment session involving two therapy applications can be used to treat most of or the entire bronchial tree 227. Substantially all of the bronchial branches extending into the lungs 211, 212 may be affected to provide a high level of therapeutic effectiveness. Because the bronchial arteries in the main bronchi 221, 222 have relatively large diameters and high heat sinking capacities, the bronchial arteries may be protected from unintended damage due to the treatment.

In some embodiments, one of the left and right main bronchi 221, 222 is treated to treat one side of the bronchial tree 227. The other main bronchus 221, 222 can be treated based on the effectiveness of the first treatment. For example, the left main bronchus 221 can be treated to treat the left lung 211. The right main bronchus 222 can be treated to treat the right lung 212. In some embodiments, a single treatment system can damage the nerve tissue of one of the bronchi 221, 222 and can damage the nerve tissue of the other main bronchus 221, 222 without removing the treatment system from the trachea 220. Nerve tissue positioned along the main bronchi 221, 222 can thus be damaged without removing the treatment system from the trachea 220. In some embodiments, a single procedure can be performed to conveniently treat substantially all, or at least a significant portion (e.g., at least 50%, 70%, 80%, 90% of the bronchial airways), of the patient's bronchial tree. In other procedures, the treatment system can be removed from the subject after treating one of the lungs 211, 212. If needed, the other lung 211, 212 can be treated in a subsequent procedure. The screening method of FIG. 1 can be performed again to determine whether additional procedures will likely provide a therapeutic effect.

The treatment system 180 can treat airways distal to the main bronchi 221, 222 can also be treated. For example, the distal tip 181 can be positioned in higher generation airways (e.g., airway generations>2) to affect remote distal portions of the bronchial tree 227. The treatment system 180 can be navigated through tortuous airways to perform a wide range of different procedures, such as, for example, denervation of a portion of a lobe, an entire lobe, multiple lobes, or one lung or both lungs. In some embodiments, the lobar bronchi are treated to denervate lung lobes. For example, one or more treatment sites along a lobar bronchus may be targeted to denervate an entire lobe connected to that lobar bronchus. Left lobar bronchi can be treated to affect the left superior lobe and/or the left inferior lobe. Right lobar bronchi can be treated to affect the right superior lobe, the right middle lobe, and/or the right inferior lobe. Lobes can be treated concurrently or sequentially. In some embodiments, a physician can treat one lobe. Based on the effectiveness of the treatment, the physician can concurrently or sequentially treat additional lobe(s). In this manner, different isolated regions of the bronchial tree can be treated.

The distal tip 181 can also be used in segmental or subsegmental bronchi. Each segmental bronchus may be treated by delivering energy to a single treatment site along each segmental bronchus. For example, energy can be delivered to each segmental bronchus of the right lung. In some procedures, ten applications of energy can treat most of or substantially all of the right lung. In some procedures, most or substantially all of both lungs are treated using less than thirty-six different applications of energy. Depending on the anatomical structure of the bronchial tree, segmental bronchi can often be denervated using one or two applications of energy.

Denervating can include damaging all of the nerve tissue of a section of a nerve trunk along an airway to stop substantially all of the signals from traveling through the damaged section of the nerve trunk to more distal locations along the bronchial tree. If a plurality of nerve trunks extends along the airway, each nerve trunk can be damaged. As such, the nerve supply along a section of the bronchial tree can be cut off. When the signals are cut off, the distal airway smooth muscle can relax, leading to airway dilation. This airway dilation reduces airflow resistance so as to increase gas exchange in the lungs 210, thereby reducing, limiting, or substantially eliminating one or more symptoms, such as breathlessness, wheezing, chest tightness, and the like. Tissue surrounding or adjacent to the targeted nerve tissue may be affected but not permanently damaged. In some embodiments, for example, the bronchial blood vessels along the treated airway can deliver a similar amount of blood to bronchial wall tissues and the pulmonary blood vessels along the treated airway can deliver a similar amount of blood to the alveolar sacs at the distal regions of the bronchial tree 227 before and after treatment. These blood vessels can continue to transport blood to maintain sufficient gas exchange. In some embodiments, airway smooth muscle is not damaged to a significant extent. For example, a relatively small section of smooth muscle in an airway wall which does not appreciably impact respiratory function may be reversibly altered. If energy is used to destroy the nerve tissue outside of the airways, a therapeutically effective amount of energy does not reach a significant portion of the non-targeted smooth muscle tissue.

Referring again to FIG. 1, bronchial thermoplasty can be performed to damage smooth muscle by ablating the entire airway wall of the bronchial tree at 164. Because the smooth muscle is destroyed, the airway can dilate. The traumatized tissue and recovery time from bronchial thermoplasty may be significantly greater than the denervation procedures disclosed in U.S. application Ser. No. 12/463,304 and U.S. application Ser. No. 12/913,702.

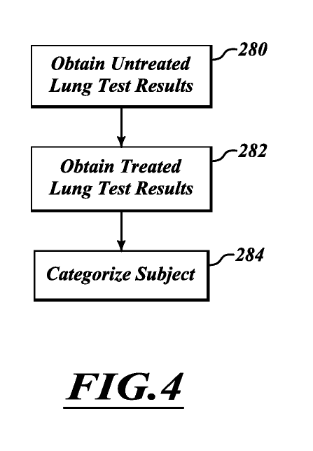

FIG. 4 shows a method of categorizing a subject. At 280, pulmonary test results from a first lung function test are obtained. A physician may administer a test (e.g., a pulmonary test) to obtain the test results. The test results may be obtained from the subject's medical file (e.g., electronic file or physical file), from testing equipment, a computing system, or the like. The subject can also perform the test outside the hospital setting. For example, a subject can perform various exercise capacity tests at home.

At 282, pulmonary test results from a second lung function test for the subject's treated lungs (e.g., treated with a test agent) are obtained. The first and the second lung function tests can be the same. Alternatively, the first and second lung function tests can be different.

At 284, the subject is categorized based on the test results into a wide range of different groups, including a candidate group, a non-candidate group, a specific treatment group, a potential candidate group, or the like. The candidate group can be comprised of subjects that will likely be responsive to therapy. The non-candidate group can be comprised of subjects that have a relatively low likelihood of receiving a therapeutic effect due to interventional therapy. The potential candidate group can be comprised of subjects that may receive a therapeutic effect in the foreseeable future. If a subject has inconsistent test results or a progressive disease, the subject can be monitored to determine whether they ultimately qualify as a candidate for interventional therapy. A specific treatment group can be comprised of subjects well suited for particular treatments. For example, one group can be subjects well suited for total lung denervation. Another group can be subjects well suited for denervation of one lung only. Yet other groups can be identified for denervation of specific lobes. Subjects in a non-denervation group may be well suited for intrabronchial valve therapy, bronchial thermoplasty, or other types of therapy. Group information can be stored by a computing system and periodically changed to reflect current treatment options.

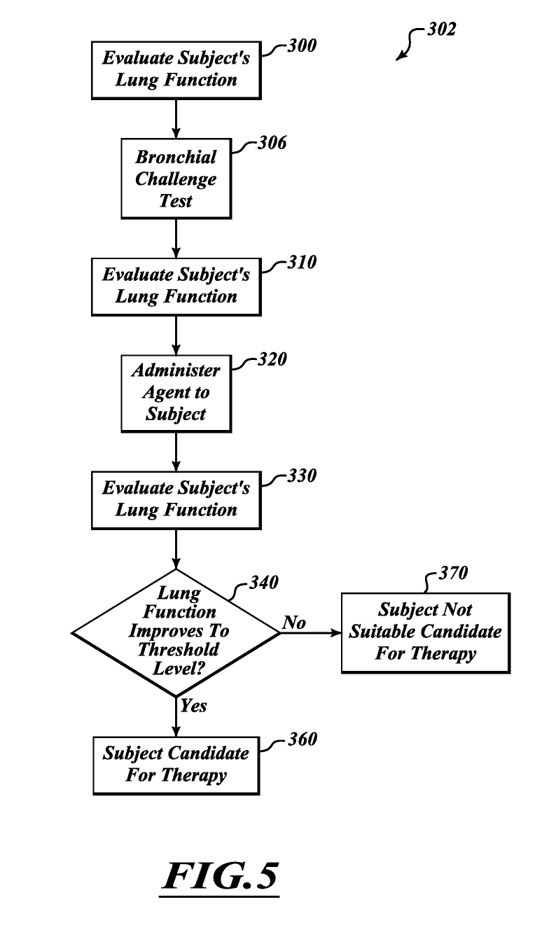

FIG. 5 shows a method 302 that involves both a bronchial challenge test and administration of an agent to predict therapy effectiveness. The method 302 may more accurately predict the responsiveness of asthma sufferers to lung denervation therapy as compared to subjects with COPD because asthma sufferers have airways that constrict when suffering an asthma attack, and are otherwise at a dilated, relatively large diameter state. The bronchial challenge test can cause bronchial constriction to simulate an asthma attack. The agent can be used to evaluate whether the constricted airway will respond positively when the nerves are ablated. Additionally, subject screening can be performed by electrical stimulation of the vagus nerve in the cervical region (i.e. neck) or other area of a patient could also be used to determine if a subject will receive a therapeutic benefit from an interventional procedure which denervates the lungs.

Subjects with COPD have airways that are chronically constricted. An anticholinergic agent without a previous bronchial challenge test can be predictive of the effectiveness of denervation therapy for enlarging the airway from its baseline constrictive state. However, if the COPD subject also has twitchy airways prone to bronchial constriction following exposure to irritants, the administration of a bronchial challenge test prior to administering the anticholinergic agent may be helpful to identify subjects whose airway irritability might be improved, even by total lung denervation and even when the airways do not enlarge from their baseline constricted state. Thus, the bronchial challenge test followed by the anticholinergic agent may indicate the potential effectiveness of denervation therapy when, in some subjects, the administration of the anticholinergic agent alone is not predictive. Details of the method 302 are discussed below.

At 300, the subject's lung function is evaluated. The evaluation can include measuring the subject's baseline lung function (e.g., FEV1 or pulmonary resistance) or other lung measurements.

At 306, airways can be constricted by performing a bronchial challenge test. One or more bronchoconstrictors can be administered. In a methacholine challenge test, a dosage of methacholine is administered to the subject. The methacholine causes narrowing of the airways. A histamine challenge test involves administering a dose of histamine. In other bronchial challenge tests, a respirator can deliver cold air to the user while the user performs various exercises. Other types of bronchial challenge tests can be utilized, if needed or desired. Bronchial challenge tests may also include electrical stimulation of the vagus nerve in the cervical region (i.e. neck) or other area of a subject.

At 310, the subject's lung function is evaluated to measure airway narrowing, if any, caused by the bronchial constriction. The subject's lung function is then evaluated. For example, FEV1 and pulmonary resistance can be measured and compared to the FEV1 and pulmonary resistance at 300.

At 320, a test agent is administered to the subject. The agents can be administered immediately after the bronchial challenge test 306 while the subject's airway is constricted. The agent can temporarily block nerve signals to the lungs and cause dilation of constricted airways. The response to the agent will thus be similar to the response during, for example, an asthma attack. The improvement in lung function can be generally proportional to the improvement in lung function that would be associated with lung denervation when there is, for example, an asthma attack.

At 330, the subject's lung function is evaluated to determine the airway dilation, if any, in response to the test agent. If the subject's airways are not dilated by the test agent, additional agents can be administered to ensure a sufficient dosage is administered.

At 340, test results from 300, 310, and/or 330 are compared to determine whether the subject is a candidate for interventional therapy. The comparison can include, without limitation, evaluating differences between test results, ratios of test results, changes in test results, or the like. In some embodiments, test results are compared with predicted test results from one or more tables.

At 360, if the subject is a candidate for therapy, the subject's medical records are updated. If the subject's lung function meets an acceptance criterion, the subject is a candidate. For example, if lung function improvement does not meet a certain threshold level from 306 to 330, the subject is identified as a non-candidate at 370. The subject can be categorized into various groups and subject status can be stored on a computing system.

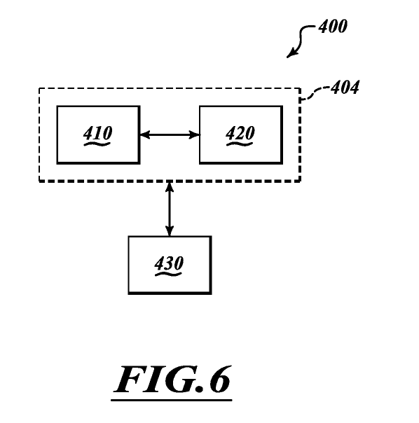

FIG. 6 shows a computing system 400 that includes a computing device 404 in communication with testing equipment 430. The computing device 404 includes a processing unit 410 that communicates with a storage device 420. The processing unit 410 can include one or more microprocessors, processors, digital signal processors (DSPs), or the like. The storage device 420 can include, without limitation, one or more hard drives, storage mediums, disks, CD-ROMs, memory, storage elements, or the like.

The equipment 430 can be in the form of respiratory testing equipment (e.g., a spirometer capable of determining lung function) and can include one or more keyboards, touch pads, scanners, communication devices, or other features for receiving information, including information from remote sources. The equipment 430 can automatically send information to the computing device 404. In some embodiments, signals are sent back and forth to optimize a testing program performed by the equipment 430.

The computing device 404 can generate protocols based upon information stored by the storage device 420. By way of example, the computing device 404 can command the testing equipment 430 based upon the subject's information (e.g., health status, age, physical fitness, or the like). During testing, the testing equipment 430 can be controlled to dynamically adjust testing.

The computing system 400 can perform many of the acts of the methods of FIGS. 1, 5, and 6. Comparisons can be automatically performed by the computing system 400 based upon information from the testing equipment 430 or information inputted by a user. Different types of programs with different algorithms or scoring systems can be used to evaluate and predict whether a subject will respond favorably to therapy. The computing system 400 can then automatically generate a recommended treatment program. For a denervation program, the computing system 400 can provide a treatment protocol that involves sequentially denervating sections of a bronchial tree. At different times during the procedure, the subject can be reevaluated to determine whether additional denervation procedures should be performed. In one embodiment, for example, after a first denervation procedure, lung function is evaluated. The most recent test results can be compared with earlier test results to evaluate whether additional denervation procedures should be performed. This process can be repeated any number of times to periodically monitor the subject during and after denervation procedures. Furthermore, subject testing during a denervation procedure can be used to optimize the denervation process to reduce or limit excessive lung denervation that may not provide a significant therapeutic effect.

Computing systems can include a wide range of different components, including, without limitation, one or more processors, microprocessors, digital signal processors (DSPs), field programmable gate arrays (FPGA), and/or application-specific integrated circuits (ASICs), memory devices, buses, power sources, and the like and can further include a processor in communication with one or more memory devices. The memories may take a variety of forms, including, for example, one or more buffers, registers, random access memories (RAMs), and/or read only memories (ROMs).

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method of evaluating a subject, comprising:
obtaining a first pulmonary test result for the subject's lungs from a first lung function test performed on the subject;
administering a test agent to the subject;
obtaining a second pulmonary test result for the subject's lungs treated with the test agent from a second lung function test performed on the subject; and
determining whether the subject's respiratory response, if any, to the test agent meets at least one acceptance criterion for denervation therapy based on a comparison of the first pulmonary test result and the second pulmonary test result.

2. The method of claim 1, further comprising:
denervating at least a portion of the subject's lungs based on the comparison of the first pulmonary test result and the second pulmonary test result.

3. The method of claim 1, further comprising:
administering the first lung function test on the subject to obtain the first pulmonary test result; and
administering the second lung function test on the subject to obtain the second pulmonary test result after the subject's lungs are treated with the test agent.

4. The method of claim 3, wherein administering the first and second lung function tests includes using a spirometer to perform spirometry.

5. The method of claim 1, further comprising categorizing the subject based on the first pulmonary test result and the second pulmonary test result.

6. The method of claim 5, wherein categorization includes identifying the subject for a candidate group for denervation or a non-candidate group for denervation.

7. The method of claim 5, wherein categorization is performed by a computing system that executes a program to determine whether the subject's respiratory response, if any, to the test agent meets at least one acceptance criterion for denervation therapy based on the comparison of the first pulmonary test result and the second pulmonary test result.

8. The method of claim 1, further comprising comparing at least one baseline lung function measurement of the first pulmonary test result to at least one lung function measurement of the second pulmonary test result.

9. The method of claim 1, further comprising:
obtaining a bronchial challenge test result from a bronchial challenge test performed on the subject; and
wherein the determination is based, at least in part, on the bronchial challenge test result.

10. The method of claim 1, further comprising determining a lung denervation protocol based on the first and second pulmonary test results.

11. The method of claim 1, further comprising applying the first lung function test to measure at least one of forced expiratory volume in 1 second for the subject, forced vital capacity for the subject, and total lung capacity for the subject.

12. The method of claim 11, further comprising allowing the subject to inhale the test agent before performing the second lung function test such that the subject's lungs are effected by the test agent.

13. The method of claim 11, wherein at least one of the first and second lung function tests includes measuring forced expiratory volume in 1 second, forced vital capacity, total lung capacity, or airway resistance of the subject.

14. The method of claim 1, further comprising denervating an airway based at least in part on a difference between the first pulmonary test result and the second pulmonary test result.

15. A method of evaluating a subject, comprising:
applying a first lung function test on the subject to obtain first information;
applying a second lung function test on the subject to obtain second information after administering an anticholinergic agent to the subject; and
comparing the first information and the second information to determine whether the subject's lung function increases to a threshold level corresponding to lung denervation therapy in response to the anticholinergic agent.

16. The method of claim 15, further comprising denervating at least a portion of a lung if the subject's lung function increases to the threshold level in response to the anticholinergic agent.

17. The method of claim 15, wherein the threshold level includes at least one of a desired threshold forced expiratory volume in 1 second for the subject, a desired threshold forced vital capacity for the subject, and a desired threshold total lung capacity for the subject.

18. A method, comprising:
evaluating a subject's lung function utilizing a bronchial challenge test;
evaluating the subject's respiratory system that has been treated with a therapeutic agent using a lung function test; and
comparing test information from the bronchial challenge test and test information from the lung function test to determine whether the subject's lung function increases to a threshold level corresponding to a lung denervation therapy in response to the therapeutic agent.

19. The method of claim 18, further comprising electrical stimulating of the vagus nerve for the bronchial challenge test.

20. The method of claim 18, further comprising performing a surgical procedure on the subject's bronchial tree or at least one of the subject's lungs based, at least in part, on the comparison.

21. The method of claim 18, wherein the threshold level is a pre-determined increase of at least one of forced expiratory volume in one second, force vital capacity, total lung capacity, and airway resistance.

22. The method of claim 18, further comprising administering the therapeutic agent which comprises a bronchodilator.

23. A system for evaluating a subject, comprising:
a storage device including first test results corresponding to a first lung function test performed on the subject and second test results corresponding to a second lung function test performed on the subject; and
a computing system configured to compare the stored first test results and the stored second test results and configured to categorize the subject based on the comparison and/or generate a denervation treatment protocol.

24. The system of claim 23, further comprising a spirometer for obtaining the first and second test results, the spirometer is in communication with the computing system.

25. The system of claim 23, wherein the computing system has circuitry configured to generate the denervation treatment protocol based on the comparison of the stored first test results and the stored second test results.
